(19) United States
(12) Patent Application Publication
(10) Pub. No.: US 2022/0130537 A1
(43) Pub. Date: Apr. 28, 2022

(54) SYSTEMS AND METHODS FOR PROVIDING HEALTH-RELATED RECOMMENDATIONS

(71) Applicant: Aegle Oy, Helsinki (FI)

(72) Inventors: Antti Pasila, Sandy Hill (AI); Kenneth Virkkunen, Helsinki (FI)

(73) Assignee: Aegle Oy, Helsinki (FI)

(21) Appl. No.: 17/082,357

(22) Filed: Oct. 28, 2020

(51) Int. Cl. G16H 50/20; G16H 10/60; G16H 40/67; G16H 10/20; G16H 50/70; G16H 20/60; G16H 20/30; G16H 40/20; G06Q 30/02; A61B 5/00; A61B 5/11

(52) U.S. Cl. CPC G16H 50/20; A61B 5/742; G16H 40/67; G16H 10/20; G16H 50/70; G16H 20/60; G16H 20/30; G16H 40/20; G06Q 30/0205; A61B 5/486; A61B 5/4866; A61B 5/7267; A61B 5/4806; A61B 5/1118; G16H 10/60

(57) ABSTRACT

A system for providing health-related recommendations. The system includes at least one server configured to receive, from user device, information indicative of value of at least one bio-signal measured for user; determine fluctuation in value of said bio-signal as function of time; detect anomaly when fluctuation in value of said bio-signal satisfies predefined criterion; send request message to user device when anomaly is detected; receive, in response to request message, information pertaining to at least one action taken by user within predefined time period prior to time instant at which value of said bio-signal is measured; determine causality between said action and value of said bio-signal; determine at least one alternative action to be recommended to user, based on determined causality; and send, to user device, recommendation message recommending said alternative action to user.

### TECHNICAL FIELD

The present disclosure relates to systems and methods for providing health-related recommendations. Furthermore, the present disclosure also relates to computer program products for providing health-related recommendations.

### BACKGROUND

In recent past, advancement in biomedical devices and associated software have provided a better quality of life to patients and carers alike. The prognosis and prescriptions by medical professionals find their basis on data generated by biomedical devices for a given patient. However, medical conditions, for example, such as diabetes and hypertension, are a major health problem globally. For prevention and management of such medical conditions, it is desirable to monitor and control bio-signals, such as a blood glucose level and a blood pressure, in a body. Notably, these bio-signals depend on parameters such as sugar intake (both amount and type thereof), salt intake, activity level (such as running, sleeping, walking, working), and genetics of a person.

It will be appreciated that living healthier lives, such as eating healthy foods and being physically active, can prevent, manage or even possibly cure such medical conditions. Various devices and application software for monitoring bio-signals and activeness of a person are known in the art. However, people often find it difficult to implement lifestyle changes required to manage their medical conditions.

Typically, various applications and devices monitor bio-signals of a person and alert the person when the measured bio-signals exceed a certain threshold. However, conventional applications and devices, such as health applications and smart watches, only measure and record bio-signals and require a human intervention to provide a feedback for the user. Furthermore, the conventional applications and devices are rule-based, and such generic rules are not designed to take into account personal preferences, behaviours and habits of o different users.

Therefore, in light of the foregoing discussion, there exists a need to overcome the aforementioned drawbacks with conventional methods of providing health-related recommendations.

### SUMMARY

The present disclosure seeks to provide a system for providing health-related recommendations. The present disclosure also seeks to provide a method for providing health-related recommendations. The present disclosure also seeks to provide a computer program product for providing health-related recommendations. An aim of the present disclosure is to provide a solution that overcomes at least partially the problems encountered in prior art.

In one aspect, an embodiment of the present disclosure provides a system for providing health-related recommendations, the system comprising at least one server configured to:
receive, from a user device of a user, information indicative of a value of at least one bio-signal measured for the user;
determine a fluctuation in the value of the at least one bio-signal as a function of time;
detect an anomaly when the fluctuation in the value of the at least one bio-signal satisfies a predefined criterion;
send a request message to the user device when the anomaly is detected;
receive, in response to the request message, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
send, to the user device, a recommendation message recommending the at least one alternative action to the user.

In another aspect, an embodiment of the present disclosure provides a method for providing health-related recommendations, the method comprising:
receiving, from a user device of a user, information indicative of a value of at least one bio-signal measured for the user;
determining a fluctuation in the value of the at least one bio-signal as a function of time;
detecting an anomaly when the fluctuation in the value of the at least one bio-signal satisfies a predefined criterion;
sending a request message to the user device when the anomaly is detected;
receiving, in response to the request message, information pertaining to at least one action taken by the user within a predefined time period prior to a time instant at which the value of the at least one bio-signal is measured;
determining a causality between the at least one action and the value of the at least one bio-signal;
determining at least one alternative action to be recommended to the user, based on the determined causality; and
sending, to the user device, a recommendation message recommending the at least one alternative action to the user.

In yet another aspect, an embodiment of the present disclosure provides a computer program product for providing health-related recommendations, the computer program product comprising a non-transitory machine-readable data storage medium having stored thereon program instructions that, when accessed by a processing device, cause the processing device to:
collect, from at least one bio-sensor, information indicative of a value of at least one bio-signal measured for a user;
determine a fluctuation in the value of the at least one bio-signal as a function of time;
detect an anomaly when the fluctuation in the value of the at least one bio-signal satisfies a predefined criterion;
display a request message when the anomaly is detected;
receive, as a user input, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
display a recommendation message recommending the at least one alternative action to the user.

Embodiments of the present disclosure substantially eliminate or at least partially address the aforementioned problems in the prior art, and enable identification of direct correlations between anomalies in bio-signals and factors causing such anomalies. Such correlations are used to provide practicable, personalized health-related recommendations for users that allow a gradual shift to healthier lifestyles.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a network environment in which a system for providing health-related recommendations is implemented, in accordance with an embodiment of the present disclosure;

FIG. 2 is a graph illustrating value of a bio-signal as a function of time, in accordance with an embodiment of the present disclosure; and

FIG. 3 is a flowchart illustrating steps of a method for providing health-related recommendations, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some o modes of carrying out the present disclosure have been disclosed, those skilled in the art would recognize that other embodiments for carrying out or practising the present disclosure are also possible.

In one aspect, an embodiment of the present disclosure provides a system for providing health-related recommendations, the system comprising at least one server configured to:
receive, from a user device of a user, information indicative of a value of at least one bio-signal measured for the user;
determine a fluctuation in the value of the at least one bio-signal as a function of time;
detect an anomaly when the fluctuation in the value of the at least one bio-signal satisfies a predefined criterion;
send a request message to the user device when the anomaly is detected;
receive, in response to the request message, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
send, to the user device, a recommendation message recommending the at least one alternative action to the user.

In another aspect, an embodiment of the present disclosure provides a method for providing health-related recommendations, the method comprising:
receiving, from a user device of a user, information indicative of a value of at least one bio-signal measured for the user;
determining a fluctuation in the value of the at least one bio-signal as a function of time;
detecting an anomaly when the fluctuation in the value of the at least one bio-signal satisfies a predefined criterion;
sending a request message to the user device when the anomaly is detected;
receiving, in response to the request message, information pertaining to at least one action taken by the user within a predefined time period prior to a time instant at which the value of the at least one bio-signal is measured;
determining a causality between the at least one action and the value of the at least one bio-signal;
determining at least one alternative action to be recommended to the user, based on the determined causality; and
sending, to the user device, a recommendation message recommending the at least one alternative action to the user.

In yet another aspect, an embodiment of the present disclosure provides a computer program product for providing health-related recommendations, the computer program product comprising a non-transitory machine-readable data storage medium having stored thereon program instructions that, when accessed by a processing device, cause the processing device to:
collect, from at least one bio-sensor, information indicative of a value of at least one bio-signal measured for a user;
determine a fluctuation in the value of the at least one bio-signal as a function of time;
detect an anomaly when the fluctuation in the value of the at least one bio-signal satisfies a predefined criterion;
display a request message when the anomaly is detected;
receive, as a user input, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
display a recommendation message recommending the at least one alternative action to the user.

The present disclosure provides the aforesaid system for providing health-related recommendations. The system enables regular monitoring of at least one bio-signal of a user and identification of causalities for any anomalies in the value of the at least one bio-signal. Based on the identified causalities, the user is provided health-related recommendations that enable the user to manage behaviour and habits thereof. Notably, the recommendations are personalised according to lifestyle and preferences of the user. Furthermore, the recommendations are tailored in a manner that allow a gradual shift towards healthier lifestyle choices made by the user. Notably, the system takes into account a possibility of the user not following the health-related recommendations if provided with an alternative action that is drastically different from his day-to-day actions, and provides a comparable alternative action as a recommendation.

Throughout the present disclosure, the term “at least one server” refers to an arrangement of physical or virtual computational entities such as a processor, and a memory unit or a database structure that includes programmable components configured to store, process and/or share information. It will be appreciated that the at least one server may be a single hardware server or a plurality of hardware servers operating in a parallel or distributed architecture. Furthermore, the at least one server comprises a database operable to store the information indicative of the value of the at least one bio-signal measured using at least one bio-sensor, fluctuations in the at least one bio-signal as a function of time and anomalies associated thereto, the predefined criterion, the at least one action reported out by the user, the at least one alternative action (recommendation), and a log of activities performed by the user. The at least one server is coupled in communication with the user device via a communication network.

Herein, the user device refers to an electronic device associated with (or used by) the user that is capable of enabling the user to perform specific tasks associated with the aforementioned system. Examples of user devices include, but are not limited to, cellular phones, smartphones, personal digital assistants (PDAs), handheld devices, laptop computers, personal computers, etc. Examples of the communication network include, but are not limited to, a cellular network, a short-range radio (for example, such as Bluetooth®) network, Internet, a wireless local area network, and an Infrared Local Area Network, or any combination thereof.

The at least one server is configured to receive, from a user device of a user, information indicative of a value of at least one bio-signal measured for the user. Herein, the term “bio-signal” refers to an electrical or non-electrical signal produced by a living being that can be measured and monitored, for example, such as heart rate, blood glucose level, oxygenation and the like, in the living being. In an example, the user device measures the at least one bio-signal for the user and provides the information indicative of the value of at least one bio-signal to the at least one server. In such example, the user device is provided with at least one bio-sensor, such as a heart rate sensor, to measure the at least one bio-signal from the user. In another example, the user device is communicably coupled to at least one bio-sensor, for example, such as a blood glucose monitor, a blood pressure monitor, a heart-rate sensor and the like, that is employed to measure the at least one bio-signal for the user.

Optionally, the at least one bio-signal is indicative of at least one of: a blood glucose level, a blood pressure, a heart rate, a heart rate variability, a body temperature, a body weight of the user. Moreover, the at least one bio-signal may be used to derive various parameters, for example, such as stress level of the user. For example, stress level of the user can be derived from heart rate variability, such that lower heart rate variability is indicative of higher stress level. The measurements are recorded repeatedly, for example such as in intervals of one second, one minute, five minutes or intermittently, or randomly. Optionally, continual monitoring of the bio-signal may be performed by employing discrete measurements indexed in a time series taken at successive equally spaced time instants. The time series data is used to extract meaningful statistics and other information indicative of the value of the at least one bio-signal. The value of the at least one bio-signal measured by the measurement device is communicated to the user device as a numeral, a graph, and the like. Subsequently, the information indicative of value of at least one bio-signal is received by the at least one server.

Moreover, the at least one server is configured to determine a fluctuation in the value of the at least one bio-signal as a function of time. It will be appreciated that the fluctuation in the value of the at least one bio-signal is a deviation in value from a previously received value of the at least one bio-signal or an average of the previously received two or more values over a period of time. Moreover, fluctuations as a function of time are indicative of a shift in the values over a period of time. In an example, the fluctuation is a single-point variation in value or a multi-fold variation.

Moreover, the at least one server is configured to detect an anomaly when the fluctuation in the value of the at least one bio-signal satisfies a predefined criterion. It will be appreciated that a slight fluctuation in the value of at least one bio-signal can be expected to be normal. However, the predefined criterion may be satisfied when the value of the at least one bio-signal deviates from its standard (normal or expected) state. Optionally, the predefined criterion is satisfied when the value of the at least one bio-signal deviates from a predefined range of the at least one bio-signal. Additionally or alternatively, optionally, the predefined criterion is satisfied when the fluctuation in the value of the at least one bio-signal exceeds a predefined fluctuation threshold of the at least one bio-signal. It will be appreciated that the predefined range and/or the predefined fluctuation threshold of the at least one bio-signal can be defined for the user in a personalized manner. Beneficially, the predefined criterion enables in identification of any abnormal fluctuation in the value of the at least one bio-signal of the user that may corresponds to a change in the user's health condition, possibly having a negative effect to the user.

The at least one server is configured to send a request message to the user device when the anomaly is detected. The request message could comprise one or more queries to be displayed at the user device. For example, the request message may be sent as a push notification to the user device. In such a case, the request message may prompt the user to answer a questionnaire comprising questions having a type selected from at least one of: single select radio button based, radio with comments, multi-select checkbox based, checkbox with comments, slider, ranking scale, rating scale, scores, drop down list, matrix grid, images, audio, free text, file upload, image upload, audio upload, and so forth. Alternatively, the request message could indicate to a software application installed on the user device to request a user input regarding the at least one action taken by the user. The user may respond to the request message by providing information in the form of a text, a selection from amongst options (on the user interface) provided to the user, an upload of an image of a food item, a video, an audio, or any combination thereof, by using the user device.

The at least one server is configured to receive, in response to the request message, information pertaining to at least one action taken by the user within a predefined time period prior to a time instant at which the value of the at least one bio-signal is measured. The user is required to respond to the request message by providing information of activities performed by the user prior to the time instant at which the value of at least one bio-signal is measured. The predefined time period could be selected as a time duration within which the at least one action taken by the user is likely to result in the fluctuation in the value of the at least one bio-signal. The predefined time period may, for example, be 1 hour, 2 hours, 3 hours, 6 hours, 12 hours, 24 hours, and so forth. The information pertaining to the at least one action is received from the user in order to establish a potential correlation between the at least one action taken by the user and the anomaly detected in the fluctuation of the at least one bio-signal.

Optionally, the information pertaining to a given action taken by a given user is indicative of at least one of: a food item consumed by the given user, an amount of the food item consumed, a physical activity performed by the given user, an amount of sleep taken by the given user. In this regard, apart from solid and semi-solid food, the food item may also include fluids, such as water, beverages, soups, and the like. The information pertaining to a given action taken by a given user may further be indicative of at least one of: a medicine consumed, an amount of the medicine, a health issue faced. In an example, information pertaining to food item(s) consumed, or the amount of food item(s) consumed may be provided manually by the user via a user interface of the user device. In another example, information pertaining to the physical activity and/or the amount of sleep may be monitored using an accelerometer and/or a global positioning system (GPS) of the user device. In such a case, the user is not required to provide such information manually.

The at least one server is configured to determine a causality between the at least one action and the value of the at least one bio-signal. The term “causality” as used herein refers to the effect of the action taken by the user within the predefined time period on the anomaly in fluctuation of the value of the at least one bio-signal. In an example, a sudden drastic increase in the value of the at least one bio-signal may be caused by a consumption of a food item attributed to such change in the value of the at least one bio-signal. It will be appreciated that causality is determined by analysing the actions done by the user in view of relation with the fluctuations in the value of the at least one bio-signal. As an example, the causality can be accounted by consumption of an amount of sugar that resulted in increased blood glucose level or consumption of an amount of salt that resulted in increased blood pressure.

The at least one server is configured to determine at least one alternative action to be recommended to the user, based on the determined causality. The causality between the at least one action taken by the user and the value of the at least one bio-signal enables recommendation of at least one alternative action, such as for example a healthier choice as an alternative to the at least one action taken by the user. The at least one alternative action is provided to the user to reinforce healthier behaviour and prevent the anomaly in fluctuation of value of the at least one least one bio-signal in future. In an example, the action taken by the user that caused an anomaly in the fluctuation to the value of the at least one bio-signal may be the user consuming a can of coke. In such example, the at least one alternative action to be recommended to the user may be consuming a can of some other aerated drink that is similar to coke in taste, but has lesser sugar content.

Optionally, the at least one alternative action comprises at least one of: a dietary substitute of a food item consumed by the user, an exercise regimen customized to the user, an appointment with a doctor. It will be appreciated that the dietary substitute of a food item consumed by the user may be different in amounts of the said food item consumed by the user, a close alternative of the said food item, or a different food item. Moreover, the exercise regimen may be for a different duration of time or a different set of exercise customized to the user. Furthermore, in an instance when the fluctuation of value of at least one bio-signal is highly abnormal, the user is advised to consult a physician. Continuing from the previous example, an alternative action recommended to the user is a can of another aerated drink that has less sugar than a can of coke. In such an example, the can of the other aerated drink is a close alternative, i.e. beverage, and a healthier choice for the user.

The at least one server is configured to send, to the user device, a recommendation message recommending the at least one alternative action to the user. The recommendation message recommending the at least one alternative action could be displayed on the user interface of the user device. In such a case, the recommendation message could be sent as a push notification. In an example, the recommendation message is provided as a text message, a set of images, an image with associated details based on the determined causality, a video or a link thereto, and so forth.

Optionally, the at least one action taken by the user comprises a food item consumed by the user, and the at least one alternative action comprises a dietary substitute of the food item recommended to the user, wherein, when determining the at least one alternative action to be recommended, the at least one server is configured to:
determine a food category to which the food item belongs;
identify a plurality of food items available locally to the user, based on a geographical location of the user; and
select the dietary substitute of the food item from amongst the plurality of food items available locally.

Optionally, in this regard, the dietary substitute of the food item is a close alternative selected from the food category. The food category may typically include, for example, fruits, vegetable, grains, meats, a junk food, a hot drink, a cool drink, a soft drink and so forth. The different types of food items are categorized into different food categories. For example, breads, noodles and pasta may be included in food category of whole wheat (or durum wheat), while cold drinks, juices, coconut water and squash may be included in soft drinks category. Moreover, information related to one or more nutritional value, such as carbohydrate, fat, protein, fibre, sugar, fructose content and the like, of the food item is also stored with the at least one server. Furthermore, the food item may further be categorised based on a nutritional value thereof. Herein, the at least one server is configured to identify a dietary substitute that belongs to the same food category and similar nutritional value as that of the food item consumed by the user. The dietary substitute of the food item is generally, a healthier alternative of the food item, for example, with lower glycaemic index or with lower sodium content (as compared to the food item consumed by the user), that when consumed may cause a lower fluctuation in the value of at least one bio-signal. The dietary substitute is identified from options available locally in the geographical location of the user, as tracked using a global positioning system of the user device. Herein, optionally, the at least one server is operable to receive information from an external server storing information related to stocks of options available locally in the geographical location of the user.

Optionally, the at least one server is configured to:
assign a similarity score to each of the plurality of food items available locally; and
select the dietary substitute of the food item based on similarity scores assigned to the plurality of food items available locally.

Optionally, in this regard, the similarity score is assigned based on the food category and at least one of: a similarity in taste, the nutritional value of the plurality of food items available locally. In an example, the similarity score is higher for food items belonging to similar food category or having similar or same nutritional value. Alternatively, the similarity score may be based on a number of other parameters such as a cumulative or average similarity score of the plurality of food items, a medical condition of the user, a time series data of the at least one action taken by the user. The similarity score is associated with a potential recommendation of the dietary substitute of the food item based on the determined causality between the at least one action taken by the user and corresponding value of the at least one bio-signal. It will be appreciated that at least one of the plurality of food items assigned a highest similarity score is selected and recommended to the user as the dietary substitute of the food item previously consumed and reported by the user. Beneficially, this encourages the user to shift slowly to healthier choices rather than an abrupt jump which may lead to loss of interest in the process of healthy lifestyle.

Optionally, the at least one server is configured to:
receive, from the user device, new information pertaining to at least one new action taken by the user;
detect, based on the new information, whether the user consumed the food item instead of the dietary substitute recommended to the user;
modify a similarity score assigned to the dietary substitute when the user consumed the food item instead of the dietary substitute; and
select a new dietary substitute of the food item from amongst remaining of the plurality of food items available locally, based on the similarity scores assigned thereto.

The user may or may not choose to consume the recommended dietary substitute. In an event when it is detected that the user has consumed the food item instead of the dietary substitute of the food item, the similarity score is reduced as the dietary substitute was not adopted by the user, therefore, indicating a dissimilarity between the food item and the dietary substitute of the food item as per user's preferences. Thereafter, a new dietary substitute is selected from amongst remaining of the plurality of food items available locally, based on the similarity scores assigned thereto.

Furthermore, optionally, the at least one server employs a machine learning model to determine the at least one alternative action to be recommended to the user. Optionally, the machine learning model is personalized to the user. In such a case, the at least one server is configured to:
receive, from the user device, information indicative of a plurality of values of the at least one bio-signal measured for the user;
receive, from the user device, information pertaining to a plurality of actions taken by the user corresponding to the plurality of values of the at least one bio-signal.
determine a causality between a given action taken by the user and a corresponding value of the at least one bio-signal measured for the user; and
train a machine learning model based on the causality between the given action and the corresponding value of the at least one bio-signal,

wherein the trained machine learning model is to be employed to determine the at least one alternative action to be recommended to the user.

Optionally, in this regard, the plurality of actions are associated with the plurality of values of the at least one bio-signal measured for a given user, and such association is used to determine the causality therebetween. Herein, the o plurality of values of the at least one bio-signal are received over a period of time in which the user used the system. Notably, the change in the plurality of values of the at least one bio-signal are indicative of general trends of the at least one bio-signal in the body of the user. In an example, a gradual increase or decrease in the plurality of values of the at least one bio-signal is indicative of a medical condition the given user is suffering from.

Moreover, the trained machine learning model employs a technique selected from at least one of: a reinforced learning, a supervised learning or an unsupervised learning. It will be appreciated that the at least one server employs federated learning techniques to train the machine learning model with the information specific to the user. In this regard, the trained machine learning model compares latest responses from the user corresponding to latest values of the at least one bio-signal with previously-received responses from the user corresponding to previous values of the at least one bio-signal to validate the causality therebetween. Based on historical patterns for the given user, the trained machine learning model determines the at least one alternative action to be recommend to the user to bring the value of the at least one bio-signal to a safe limit defined for the at least one bio-signal. Notably, the at least one server is configured to store the plurality of values of the at least one bio-signal, the plurality of actions taken by the user corresponding thereto, the causality determined between a given action taken by the user and a corresponding value of the at least one bio-signal measured for the user, and the at least one alternative action to be recommended to the user. In an example, the trained machine learning model may be employed to recommend the user to limit consumption of tea to one cup per day based on the determined causality of tea consumption on the value of the blood sugar levels of the user based on the response thereof during past 1 week, for example. Furthermore, over a period of time, the machine learning model learns certain information about habits and preferences of the user that is employed in determining the at least one alternative action. For example, the user may be habitually averse to not reducing high amounts of sugary drinks in his daily consumption. In such example, the trained machine learning model may recommend an increase in physical activity to offset the calories of sugary drinks instead of recommending the user to replace sugary drinks. Furthermore, the machine learning model is configured to employ some ‘universal’ impacts such as direct correlations between the value of the at least one bio-signal, such as an increase in the blood glucose level, and the corresponding action taken by the users, such as an amount of sugar, to be trained efficiently and faster for an individual. Beneficially, the trained machine learning model enables monitoring and maintaining the values of the at least one bio-signal of the user customized based on the measurement data (data sets) provided by the user device and the response to request message by the user.

Additionally, optionally, the at least one server is configured to:
receive, from a plurality of user devices of a plurality of users, information indicative of a plurality of values of the at least one bio-signal measured for corresponding users;
receive, from the plurality of user devices, information pertaining to corresponding actions taken by the plurality of users corresponding to the plurality of values of the at least one bio-signal;
determine a causality between an action taken by a given user and a corresponding value of the at least one bio-signal measured for the given user; and
train the machine learning model based on the causality between the action taken by the given user and the corresponding value of the at least one bio-signal measured for the given user.

Optionally, in this regard, the information relating to the plurality of users is used to identify insights from a broader sample set and to train the machine learning model accordingly based on behaviours and preferences of the broader sample set. Herein, the plurality of users may belong to a same demographic as the aforementioned user. It will be appreciated that regional and cultural preferences of the users may contribute to training the machine learning model for a generalized use. Therefore, selecting the plurality of users from a given demographic may assist in providing relevant recommendations to the user. Herein, the machine learning model is configured to take into account an individual behaviour as a first part of the training data and a group behaviour from the plurality of users as a second part of the training data.

In an exemplary implementation, the system provides health-related recommendations to diabetic patients. The recommendations are provided to encourage the users of the system to slowly be accustomed to healthier choices. The system uses a glucose measurement sensor as a bio-sensor for measuring value of the blood glucose level in the body routinely, for example, such as every 5 minutes. The glucose measurement sensor is communicably coupled to a user device associated with the user configured to process the measurement data or provide the measurement data to the at least one server for processing thereof. As an example, the measurement data is analyzed to detect fluctuation in the blood glucose level from a predefined range, for example, such as in a range between 3.9 mmol/L and 7.1 mmol/L. The fluctuation in the blood glucose level is used to determine any associated anomaly and a notification is displayed on the smartphone to receive input from the user regarding recent activities done that resulted in such fluctuation. As another example, if the blood glucose level increases from 5 mmol/L to 9 mmol/L over a period of 15 minutes, a request message is rendered for the user to respond to via the user device. The request message may, for example, be ‘What did you eat during in last 60 minutes?’ and the user response may, for example, be ‘Can of Coca Cola’. Therefore, the at least one server is configured to convert the amount of Coca Cola serving, i.e. 375 ml, to an amount of sugar, i.e. 39 g, in the said serving. It will be appreciated that said conversion is attributed to the role of sugar in the change of the blood glucose level in the body of the user. Notably, the impact of the said serving on the blood glucose level is measured as a function of time. Furthermore, the at least one action taken by the user corresponding to the value of the blood glucose level as a function of time is optionally used to train a machine learning model, based on, for example, reinforcement learning algorithms. Such training of the machine learning model enables recommending an alternative action, such as a substitute for a ‘Coca Cola’, for example any one of: ‘Pepsi’, ‘Sugar Free Coca Cola’, ‘Apple Juice’, or another similar beverage locally available to the user based on a score assigned to each of these dietary substitutes. Thus, the at least one server recommends to the user, for example, a can of ‘Pepsi’ as a substitute to the can of ‘Coca Cola’ with a notification, for example, ‘Next time try to have Pepsi instead of Coca Cola. It might reduce rising of the blood sugar level that high’. The at least one server is further configured to monitor the effects of the available dietary substitutes over a period of time and observe, based thereon, for example, that blood glucose level did not peak to 9 mmol/L but to 8 mmol/L if the said serving of ‘Coca Cola’ is replaced with a serving of ‘Pepsi’, i.e. 375 ml. The at least one server may be further be configured to follow-up with the user in the next measurement cycle to check if the recommendation has been followed. In this regard, if the user does not choose to follow the recommendation, the at least one server modifies the similarity score between ‘Coca Cola’ and ‘Pepsi’, and does not recommend Pepsi in the future, instead recommends another dietary substitute, for example such as ‘Sugar-free Coca Cola’.

The present disclosure also relates to the method as described above. Various embodiments and variants disclosed above apply mutatis mutandis to the method.

Optionally, the method further comprises:
receiving, from the user device, information indicative of a plurality of values of the at least one bio-signal measured for the user;
receiving, from the user device, information pertaining to a plurality of actions taken by the user corresponding to the plurality of values of the at least one bio-signal;
determining a causality between a given action taken by the user and a corresponding value of the at least one bio-signal measured for the user; and
training a machine learning model based on the causality between the given action and the corresponding value of the at least one bio-signal,

wherein the trained machine learning model is employed to determine the at least one alternative action to be recommended to the user.

Additionally, optionally, the method further comprises:
receiving, from a plurality of user devices of a plurality of users, information indicative of a plurality of values of the at least one bio-signal measured for corresponding users;
receiving, from the plurality of user devices, information pertaining to corresponding actions taken by the plurality of users corresponding to the plurality of values of the at least one bio-signal;
determining a causality between an action taken by a given user and a corresponding value of the at least one bio-signal measured for the given user; and
training the machine learning model based on the causality between the action taken by the given user and the corresponding value of the at least one bio-signal measured for the given user.

Optionally, the at least one action taken by the user comprises a food item consumed by the user, and the at least one alternative action comprises a dietary substitute of the food item recommended to the user, wherein the step of determining the at least one alternative action to be recommended comprises:
determining a food category to which the food item belongs;
identifying a plurality of food items available locally to the user, based on a geographical location of the user; and
selecting the dietary substitute of the food item from amongst the plurality of food items available locally.

Optionally, the method further comprises assigning a similarity score to each of the plurality of food items available locally, wherein the step of selecting the dietary substitute of the food item is performed based on similarity scores assigned to the plurality of food items available locally.

Optionally, the method further comprises:
receiving, from the user device, new information pertaining to at least one new action taken by the user;
detecting, based on the new information, whether the user consumed the food item instead of the dietary substitute recommended to the user;
modifying a similarity score assigned to the dietary substitute when the user consumed the food item instead of the dietary substitute; and
selecting a new dietary substitute of the food item from amongst remaining of the plurality of food items available locally, based on the similarity scores assigned thereto.

The present disclosure also relates to the computer program product as described above. Various embodiments and variants disclosed above apply mutatis mutandis to the computer program product.

The processing device is coupled in communication with the at least one bio-sensor. Optionally, the program instructions are downloadable from a software application store, for example, such as an “App store” to the processing device.

Optionally, the program instructions cause the processing device to:
collect, from the at least one bio-sensor, information indicative of a plurality of values of the at least one bio-signal measured for the user;
receive, as user inputs, information pertaining to a plurality of actions taken by the user corresponding to the plurality of values of the at least one bio-signal;
determine a causality between a given action taken by the user and a corresponding value of the at least one bio-signal measured for the user; and
train a machine learning model based on the causality between the given action and the corresponding value of the at least one bio-signal,

wherein the trained machine learning model is employed to determine the at least one alternative action to be recommended to the user.

Optionally, the at least one action taken by the user comprises a food item consumed by the user, and the at least one alternative action comprises a dietary substitute of the food item recommended to the user, wherein, when determining the at least one alternative action to be recommended, the program instructions cause the processing device to:
determine a food category to which the food item belongs;
identify a plurality of food items available locally to the user, based on a geographical location of the user; and
select the dietary substitute of the food item from amongst the plurality of food items available locally.

Optionally, the program instructions cause the processing device to:
assign a similarity score to each of the plurality of food items available locally; and
select the dietary substitute of the food item based on similarity scores assigned to the plurality of food items available locally.

Optionally, the program instructions cause the processing device to:
receive, as a user input, new information pertaining to at least one new action taken by the user;
detect, based on the new information, whether the user consumed the food item instead of the dietary substitute recommended to the user;
modify a similarity score assigned to the dietary substitute when the user consumed the food item instead of the dietary substitute; and
select a new dietary substitute of the food item from amongst remaining of the plurality of food items available locally, based on the similarity scores assigned thereto.

### DETAILED DESCRIPTION OF THE DRAWINGS

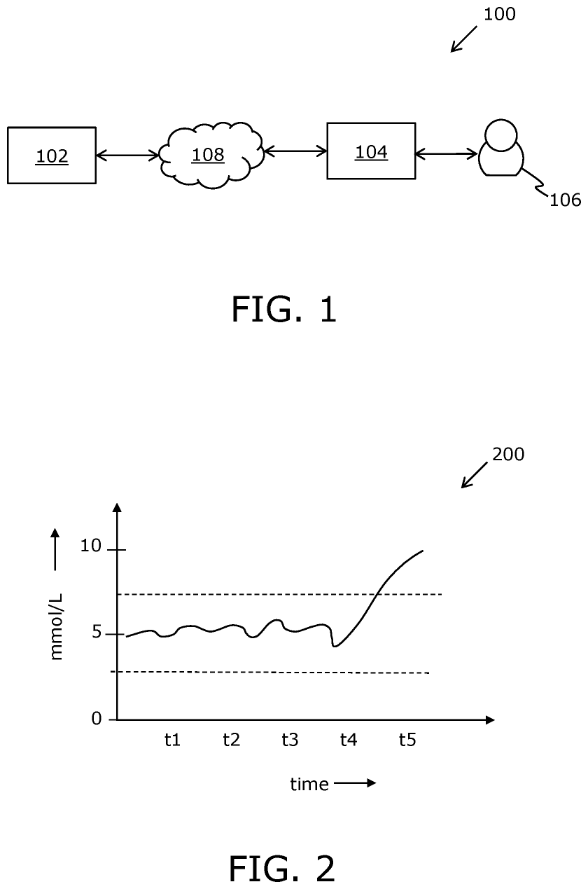

Referring to FIG. 1, illustrated is a schematic illustration of a network environment 100 in which a system for providing health-related recommendations can be implemented, in accordance with an embodiment of the present disclosure. As shown, the system comprises at least one server 102 coupled in communication with a user device 104 (associated with a user 106) via a communication network 108. The at least one server 102 is configured to:
receive, from the user device 104 of the user 106, information indicative of a value of at least one bio-signal measured for the user 106;
determine a fluctuation in the value of the at least one bio-signal as a function of time;
detect an anomaly when the fluctuation in the value of the at least one bio-signal satisfies a predefined criterion;
send a request message to the user device 104 when the anomaly is detected;
receive, in response to the request message, information pertaining to at least one action taken by the user 106 within a predefined time period prior to a time instant at which the value of the at least one bio-signal is measured;
determine a causality between the at least one action and the value of the at least one bio-signal;
determine at least one alternative action to be recommended to the user 106, based on the determined causality; and
send, to the user device 104, a recommendation message recommending the at least one alternative action to the user 106.

Referring to FIG. 2, illustrated is a graph 200 illustrating value of a bio-signal as a function of time, in accordance with an embodiment of the present disclosure. As shown, the value of the bio-signal, represented as y-axis, is measured as a function of time, represented as x-axis, at time instances t1, t2, t3, t4, t5, at regular time intervals, for example, after every 5 minutes. Notably, a fluctuation in the value of the bio-signal, i.e. from 5 mmol/L to 9 mmol/L is detected in a time period between t3 to t5, i.e. in 10 minutes. Subsequently, a request message is sent to a user device when an anomaly in the value of the bio-signal is detected.

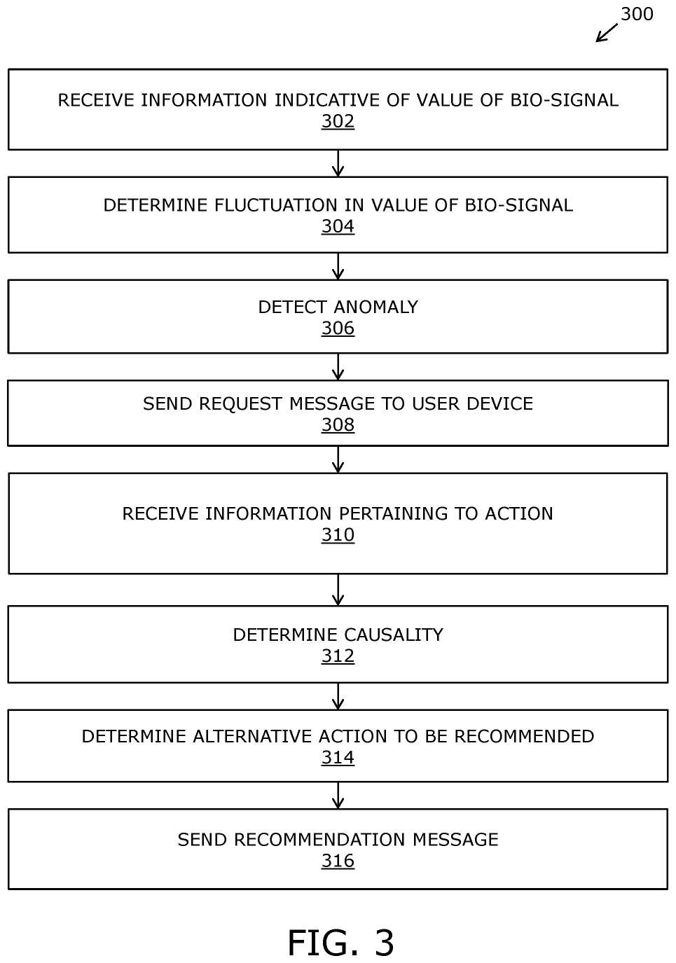

Referring to FIG. 3, there is shown a flowchart 300 illustrating steps of a method for providing health-related recommendations, in accordance with an embodiment of the present disclosure. At step 302, information indicative of a value of at least one bio-signal measured for the user is received from a user device of a user. At step 304, a fluctuation in the value of the at least one bio-signal is determined as a function of time. At step 306, an anomaly is detected when the fluctuation in the value of the at least one bio-signal satisfies a predefined criterion. At step 308, a request message is sent to the user device when the anomaly is detected. At step 310, information pertaining to at least one action taken by the user within a predefined time period prior to a time instant at which the value of the at least one bio-signal is measured is received in response to the request message. At step 312, a causality between the at least one action and the value of the at least one bio-signal is determined. At step 314, at least one alternative action to be recommended to the user is determined based on the determined causality. At step 316, a recommendation message recommending the at least one alternative action to the user is sent to the user device.

The steps 302, 304, 306, 308, 310, 312, 314 and 316 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for providing health-related recommendations, the system comprising at least one server configured to:
receive, from a user device of a user, information indicative of a value of at least one bio-signal measured for the user;
determine a fluctuation in the value of the at least one bio-signal as a function of time;
detect an anomaly when the fluctuation in the value of the at least one bio-signal satisfies a predefined criterion;
send a request message to the user device when the anomaly is detected;
receive, in response to the request message, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
send, to the user device, a recommendation message recommending the at least one alternative action to the user.

2. The system of claim 1, wherein the at least one server is configured to:
receive, from the user device, information indicative of a plurality of values of the at least one bio-signal measured for the user;
receive, from the user device, information pertaining to a plurality of actions taken by the user corresponding to the plurality of values of the at least one bio-signal;
determine a causality between a given action taken by the user and a corresponding value of the at least one bio-signal measured for the user; and
train a machine learning model based on the causality between the given action and the corresponding value of the at least one bio-signal, wherein the trained machine learning model is to be employed to determine the at least one alternative action to be recommended to the user.

3. The system of claim 2, wherein the at least one server is configured to:
receive, from a plurality of user devices of a plurality of users, information indicative of a plurality of values of the at least one bio-signal measured for corresponding users;
receive, from the plurality of user devices, information pertaining to corresponding actions taken by the plurality of users corresponding to the plurality of values of the at least one bio-signal;
determine a causality between an action taken by a given user and a corresponding value of the at least one bio-signal measured for the given user; and
train the machine learning model based on the causality between the action taken by the given user and the corresponding value of the at least one bio-signal measured for the given user.

4. The system of any of the preceding claims 1, wherein the information pertaining to a given action taken by a given user is indicative of at least one of: a food item consumed by the given user, an amount of the food item consumed, a physical activity performed by the given user, an amount of sleep taken by the given user.

5. The system of claim 1, wherein the at least one alternative action comprises at least one of: a dietary substitute of a food item consumed by the user, an exercise regimen customized to the user, an appointment with a doctor.

6. The system of claim 1, wherein the at least one action taken by the user comprises a food item consumed by the user, and the at least one alternative action comprises a dietary substitute of the food item recommended to the user, wherein, when determining the at least one alternative action to be recommended, the at least one server is configured to:
determine a food category to which the food item belongs;
identify a plurality of food items available locally to the user, based on a geographical location of the user; and
select the dietary substitute of the food item from amongst the plurality of food items available locally.

7. The system of claim 6, wherein the at least one server is configured to:
assign a similarity score to each of the plurality of food items available locally; and
select the dietary substitute of the food item based on similarity scores assigned to the plurality of food items available locally.

8. The system of claim 7, wherein the at least one server is configured to:
receive, from the user device, new information pertaining to at least one new action taken by the user;
detect, based on the new information, whether the user consumed the food item instead of the dietary substitute recommended to the user;
modify a similarity score assigned to the dietary substitute when the user consumed the food item instead of the dietary substitute; and
select a new dietary substitute of the food item from amongst remaining of the plurality of food items available locally, based on the similarity scores assigned thereto.

9. A method for providing health-related recommendations, the method comprising:
receiving, from a user device of a user, information indicative of a value of at least one bio-signal measured for the user;
determining a fluctuation in the value of the at least one bio-signal as a function of time;
detecting an anomaly when the fluctuation in the value of the at least one bio-signal satisfies a predefined criterion;
sending a request message to the user device when the anomaly is detected;
receiving, in response to the request message, information pertaining to at least one action taken by the user within a predefined time period prior to a time instant at which the value of the at least one bio-signal is measured;
determining a causality between the at least one action and the value of the at least one bio-signal;
determining at least one alternative action to be recommended to the user, based on the determined causality; and
sending, to the user device, a recommendation message recommending the at least one alternative action to the user.

10. The method of claim 9, further comprising:
receiving, from the user device, information indicative of a plurality of values of the at least one bio-signal measured for the user;
receiving, from the user device, information pertaining to a plurality of actions taken by the user corresponding to the plurality of values of the at least one bio-signal;
determining a causality between a given action taken by the user and a corresponding value of the at least one bio-signal measured for the user; and
training a machine learning model based on the causality between the given action and the corresponding value of the at least one bio-signal, wherein the trained machine learning model is employed to determine the at least one alternative action to be recommended to the user.

11. The method of claim 10, further comprising:
receiving, from a plurality of user devices of a plurality of users, information indicative of a plurality of values of the at least one bio-signal measured for corresponding users;
receiving, from the plurality of user devices, information pertaining to corresponding actions taken by the plurality of users corresponding to the plurality of values of the at least one bio-signal;
determining a causality between an action taken by a given user and a corresponding value of the at least one bio-signal measured for the given user; and
training the machine learning model based on the causality between the action taken by the given user and the corresponding value of the at least one bio-signal measured for the given user.

12. The method of claim 9, wherein the at least one action taken by the user comprises a food item consumed by the user, and the at least one alternative action comprises a dietary substitute of the food item recommended to the user, wherein the step of determining the at least one alternative action to be recommended comprises:
determining a food category to which the food item belongs;
identifying a plurality of food items available locally to the user, based on a geographical location of the user; and
selecting the dietary substitute of the food item from amongst the plurality of food items available locally.

13. The method of claim 12, further comprising assigning a similarity score to each of the plurality of food items available locally, wherein the step of selecting the dietary substitute of the food item is performed based on similarity scores assigned to the plurality of food items available locally.

14. The method of claim 13, further comprising:
receiving, from the user device, new information pertaining to at least one new action taken by the user;
detecting, based on the new information, whether the user consumed the food item instead of the dietary substitute recommended to the user;
modifying a similarity score assigned to the dietary substitute when the user consumed the food item instead of the dietary substitute; and
selecting a new dietary substitute of the food item from amongst remaining of the plurality of food items available locally, based on the similarity scores assigned thereto.

15. A computer program product for providing health-related recommendations, the computer program product comprising a non-transitory machine-readable data storage medium having stored thereon program instructions that, when accessed by a processing device, cause the processing device to:
collect, from at least one bio-sensor, information indicative of a value of at least one bio-signal measured for a user;
determine a fluctuation in the value of the at least one bio-signal as a function of time;
detect an anomaly when the fluctuation in the value of the at least one bio-signal satisfies a predefined criterion;
display a request message when the anomaly is detected;
receive, as a user input, information pertaining to at least one action taken by the user within a predefined time period prior to a time instant at which the value of the at least one bio-signal is measured;
determine a causality between the at least one action and the value of the at least one bio-signal;
determine at least one alternative action to be recommended to the user, based on the determined causality; and
display a recommendation message recommending the at least one alternative action to the user.

16. The computer program product of claim 15, wherein the program instructions cause the processing device to:
collect, from the at least one bio-sensor, information indicative of a plurality of values of the at least one bio-signal measured for the user;
receive, as user inputs, information pertaining to a plurality of actions taken by the user corresponding to the plurality of values of the at least one bio-signal;
determine a causality between a given action taken by the user and a corresponding value of the at least one bio-signal measured for the user; and
train a machine learning model based on the causality between the given action and the corresponding value of the at least one bio-signal, wherein the trained machine learning model is employed to determine the at least one alternative action to be recommended to the user.

17. The computer program product of claim 15, wherein the at least one action taken by the user comprises a food item consumed by the user, and the at least one alternative action comprises a dietary substitute of the food item recommended to the user, wherein, when determining the at least one alternative action to be recommended, the program instructions cause the processing device to:
determine a food category to which the food item belongs;
identify a plurality of food items available locally to the user, based on a geographical location of the user; and
select the dietary substitute of the food item from amongst the plurality of food items available locally.

18. The computer program product of claim 17, wherein the program instructions cause the processing device to:
assign a similarity score to each of the plurality of food items available locally; and
select the dietary substitute of the food item based on similarity scores assigned to the plurality of food items available locally.

19. The computer program product of claim 18, wherein the program instructions cause the processing device to:
receive, as a user input, new information pertaining to at least one new action taken by the user;
detect, based on the new information, whether the user consumed the food item instead of the dietary substitute recommended to the user;
modify a similarity score assigned to the dietary substitute when the user consumed the food item instead of the dietary substitute; and
select a new dietary substitute of the food item from amongst remaining of the plurality of food items available locally, based on the similarity scores assigned thereto.
